(19) United States
(12) Patent Application Publication
(10) Pub. No.: US 2020/0258609 A1
(43) Pub. Date: Aug. 13, 2020

(54) MEDICAL COMMUNICATION AND MANAGEMENT PLATFORM

(71) Applicants: Darren MCMASTER, San Antonio, TX (US); Stephen Trey MOORE, San Antonio, TX (US); Careteam.io, Inc., San Antonio, TX (US)

(72) Inventors: Darren McMaster, San Antonio, TX (US); Stephen Trey Moore, San Antonio, TX (US)

(21) Appl. No.: 16/753,733

(22) PCT Filed: Oct. 5, 2018
(86) PCT No.: PCT/US2018/054694

§ 371 Date: Apr. 3, 2020

Related U.S. Application Data
(60) Provisional application No. 62/569,361, filed on Oct. 6, 2017.

(51) Int. Cl. G16H 15/00; G16H 10/60; G16H 50/70; G16H 50/20; G16H 10/20; G16H 50/30; G16H 70/20; G16H 40/20; G16H 40/67

(52) U.S. Cl. CPC G16H 15/00; G16H 10/60; G16H 50/70; G16H 50/20; G16H 40/67; G16H 50/30; G16H 70/20; G16H 40/20; G16H 10/20

(57) ABSTRACT

Implementations of the present disclosure include methods, systems, and computer-readable storage mediums for tracking and communicating health condition of patients. In some implementations, a medical procedure associated with a patient is received. A plurality of patient records associated with the medical procedure are obtained from a database. The plurality of patient records includes medical histories of a plurality of patients. A questionnaire is generated by applying machine learning techniques on the plurality of patient records. The questionnaire is provided to the patient. A set of answers for the questionnaire is received. A health score is calculated for the patient based on the set of answers and the plurality of patient records. Based on the health score, a health notification associated with the patient is provided.

### CROSS REFERENCE TO RELATED APPLICATIONS

This application claims the benefit of U.S. Provisional Ser. No. 62/569,361, filed Oct. 6, 2017. The disclosure of the prior application is considered part of and is incorporated by reference in the disclosure of this application.

### BACKGROUND

Millions of surgeries are performed every year in medical service environments. Each surgery generally involves a large number of medical personnel, whose schedules must be coordinated to perform the surgical procedure. Timing of the procedure is also critical in many instances, and the coordination may also need to accommodate various other resources such as facilities, equipment, and so forth. Accordingly, the coordination of a surgical procedure is a challenge, as is managing the pre-operation and post-operation collection and dissemination of information to the patient and/or medical personnel. In order to effectively monitor a patient's status following an operation, health care team members, such as physicians and nurses, must spend a substantial amount of time calling the patient and meeting with the patient at office visits.

### SUMMARY

Implementations of the present disclosure are generally directed to managing and communicating medical information, such as information related to pre-operative or post-operative patient care. More specifically, implementations provide a platform that monitors the patients and notifies the medical staff about the health conditions of the patients.

In general, innovative aspects of the subject matter described in this specification can be embodied in methods that includes operations of: receiving patient information comprising a medical procedure associated with a patient; obtaining, from a database, a plurality of patient records associated with the medical procedure, the plurality of patient records including medical histories of a plurality of patients; providing a questionnaire to the patient, wherein the questionnaire comprises a set of questions that is generated by applying a machine learning technique on the plurality of patient records; receiving a set of answers for the set of questions; calculating a health score for the patient, the health score being calculated based on the set of answers and the plurality of patient records; providing, based on the health score, a health notification associated with the patient, the health notification is being provided to be displayed on a computing device.

Implementations can optionally include one or more of the following features: the health notification includes a prediction of a health risk that is to happen to the patient at a future time; the set of questions are generated based on one or more trends identified in one or more health categories associated with the plurality of patient records, the one or more trends being determined by applying the machine learning technique on the plurality of patient records; the health score is calculated by comparing the set of answers to the one or more trends; the health notification is provided in response to determining that the health score is more than a predefined threshold value; the set of questions are directed to one or more health categories and the health score is calculated based on an aggregation of one or more sub-scores associated with the one or more health categories; the health notification is provided in response to determining that a sub-score of the one or more sub-scores is beyond a predefined range associated with a respective health category that corresponds with the sub-score; the health notification is provided to a medical staff whose specialty is associated with the respective health category; at least one health category of the one or more health categories is associated with at least one of a pain level, physical functions, a history of medications consumption, level of appetite, and vital signs of the patient; each question in the set of questions is directed to one health category of the one or more health categories; each sub-score is associated with one answer in the set of answers; the patient is associated with one or more medical staff, and the computing device on which the health notification is being displayed is associated with at least one of the one or more medical staff; the health notification is provided to an on-call medical staff whose specialty is related to the medical procedure; the medical procedure includes one or more procedures based on which the patient is currently being treated; the medical procedure includes post-surgical treatment procedures; the medical procedure includes pre-surgical treatment procedures; actions further include automatically scheduling one or more treatment procedures based on the health score and the medical histories in the plurality of patient records.

Other implementations of any of the above aspects include corresponding systems, apparatus, and computer programs that are configured to perform the actions of the methods, encoded on computer storage devices. The present disclosure also provides a computer-readable storage medium coupled to one or more processors and having instructions stored thereon which, when executed by the one or more processors, cause the one or more processors to perform operations in accordance with implementations of the methods provided herein.

The present disclosure further provides a system for implementing the methods provided herein. The system includes one or more processors, and a computer-readable storage medium coupled to the one or more processors having instructions stored thereon which, when executed by the one or more processors, cause the one or more processors to perform operations in accordance with implementations of the methods provided herein.

Implementations of the present disclosure provide one or more of the following technical advantages and/or technical improvements over previously available solutions. Limitations on health providers' time makes effective monitoring of the patients' health a challenge. For example, an improper monitoring of a patient's post-surgery health may increase the risk of the patient experiencing a medical emergency following the surgery. By providing a system for automatically monitoring and reporting patients' health status, the present disclosure reduces the risk of patients experiencing medical emergencies. The implementations of the present disclosure provide for effective monitoring of patients during a medical treatment procedure, for example, post-operative progress of patient.

Current means of patient monitoring are often limited to tracking of vital signs monitored by medical devices, which may not effectively indicate the risk of certain post-operative complications. The present disclosure employs the use of question-and-answer feedback obtained directly from the patient, and uses this information to generate a health score that is more closely aimed at detecting potential post-operative complications than traditional vital sign monitoring. The question-and-answer feedbacks can be descriptive and provide more information about the overall or particular details of the patient's health condition. This improved form of monitoring results in faster identification of complications, which reduces post-operative emergencies.

Traditionally, a patient would have to be in a hospital or other medical care centers in order for the medical staff to monitor the patient's medical condition. With implementations of the present disclosure, the patients do not need to be in a hospital or other medical care centers in order to provide the real-time monitoring of their health condition, as the patient can provide information regarding her medical condition from any location using their personal computing devices that are in communication with the medical platform disclosed herein.

The implementations disclosed herein are capable of determining medical treatment procedures that should be performed on the patients based on the health condition of the respective patients. The implementations can automatically schedule for the medical treatment procedure. Accordingly, the implementations can save lives of the patients, and time and energy of the medical staff.

The medical platform disclosed herein improves and streamlines the workflows, processes, and communication that occurs among surgical care team members, such as surgeons, MAs, surgical coordinators (SCs), first assists, anesthesiologists, medical sales representatives, and so forth. The platform allows care team coordination through a shared calendar, and is designed to improve how the MA and/or SC communicates with the surgeon and the entire care team, as well as confirming the surgery time and location with their patients. The platform also allows for communication with the patient, including not only the ability to chat but the automated sending of notifications and reminders to the patient for all pre-op checklists, including billing and payment collection. After the procedure, the patient uses the platform to clearly understand their post-op instructions, order supplies and ask questions of the care team as well as posting their progress (including images when appropriate). The platform allows the surgeon and MA to closely and efficiently follow their patient's recovery in near real time, without the need to make phone calls.

It is appreciated that methods in accordance with the present disclosure can include any combination of the aspects and features described herein. That is, methods in accordance with the present disclosure are not limited to the combinations of aspects and features specifically described herein, but also include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

FIG. 1 depicts an example platform, according to implementations of the present disclosure.

FIGS. 2A-2C, 3A-3C, 4, 5A-5B, 6A-6E, and 7A-7C depict example user interfaces, according to implementations of the present disclosure.

FIG. 8 depicts an example process that can be executed in accordance with implementations of the present disclosure.

FIG. 9 depicts an example computing system, according to implementations of the present disclosure.

### DETAILED DESCRIPTION

Implementations of the present disclosure are directed to systems, devices, methods, and computer-readable media for tracking the medical condition of the patients and notifying appropriate medical staff about the medical conditions of the patients.

The implementations can manage and communicate information regarding medical procedures based on which a patient is being treated. Examples of the medical procedures include, but not is limited to, post-surgical treatments and pre-surgical treatments.

The Implementations provide a software platform that executes on one or more computing devices. The platform facilitating the management and communication of information regarding the medical procedures associated with the respective patients. The is capable of monitoring the patient's health condition and reporting the health condition to the patient and/or one or more medical staff associated with the patient (e.g., a physician who has performed a surgery on the patient).

The platform provides various user interfaces (UI) that users can access to view and update information relevant to a procedure. For example, a medical assistant (MA) can act as a surgical coordinator, and can use the platform to coordinate all the tasks to be performed, information to be gathered, and personnel, facilities, and/or equipment to be scheduled in order for the surgery to be performed at a scheduled time.

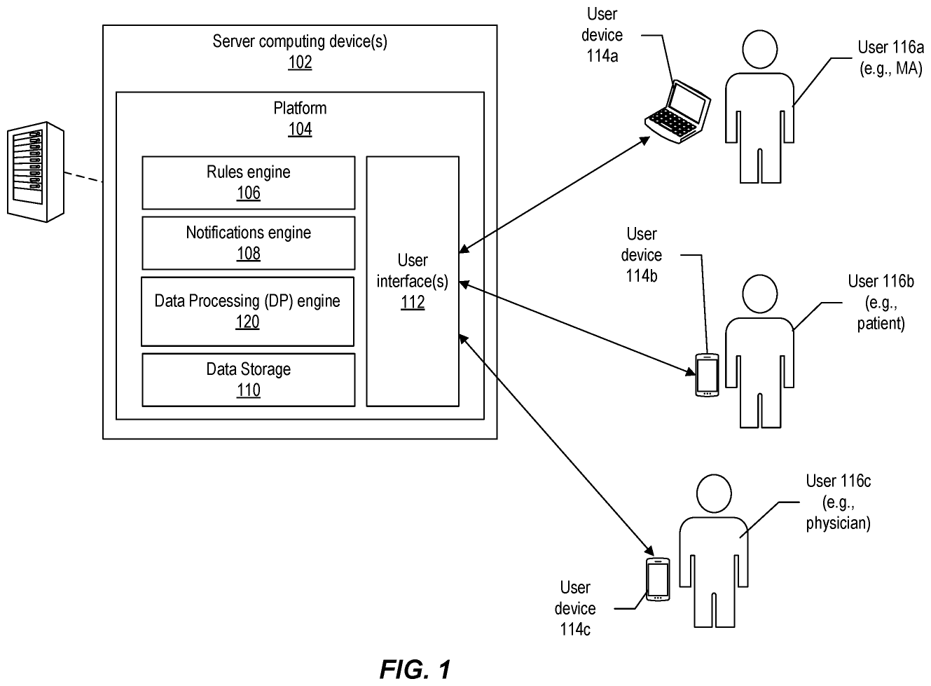

FIG. 1 depicts an example platform, according to the implementations of the present disclosure. The platform 104 executes, as software, on one or more server computing devices 102. The device(s) 102 can include any appropriate number and type of computing device. The platform 104 can include a rules engine 106, a notifications engine 108, data processing engine 120, data storage 110, and UI(s) 112.

The rules engine 106 can apply rules to determine, based on a specified type of procedure, the various requirements for the procedure, including personnel, equipment, notifications, medications, and so forth. The notifications engine 108 can operate to send notifications to users of the platform 104, such as patient 116b, physician 116c, MA 116a, and so forth.

The data storage 110 stores data relevant to the operations of the platform, such as patients information describing the patients, procedures information describing the procedures, team members information regarding the particular team members associated with a surgery, and so forth. The data storage 110 can store data using any suitable data storage format. The UI(s) 112 provides various views into the information regarding a surgical procedure, as described further below.

Various types of users 116, such as a patient, an MA, other medical team members (e.g., physician), and so forth may access the UI(s) 112 according to their roles and the access permissions associated with their roles. The users 116 may employ user devices 114 to access the UI(s) 112. The user device(s) 114 can include any suitable type of computing device, including portable devices (e.g., smartphone, tablet computer, etc.) and/or less portable devices (e.g., desktop computer, laptop computer, etc.). The platform can support various types of UI(s) 112. For example, the UI(s) may be rendered in applications (apps) executing on the user device(s) 114. The UI(s) may also be rendered as web page(s) in browsers executing on the user device(s) 114, the web page(s) served from web server(s), app server(s), and so forth executing on the device(s) 102.

The platform can be used to track a patient's health conditions. In some implementations, the platform 104 is used to track patient outcomes following a medical procedure. The platform 104 receives medical information associated with the patient 116b (also referred to as “the medical procedure of the patient 116b,” herein) from the one or more user devices, e.g., the user device 114a, 114b, and/or 114c. The medical information can include one or more medical procedures for which user patient 116b is currently being treated. The medical information can include data regarding the type of medical procedure performed or to be performed on the user patient 116b.

The medical procedure of patient 116b can include, but is not limited to, information related to pre-operative treatment procedures, surgical procedures, and post-operative treatment procedures. In some implementations, the information regarding the type of medical procedure performed may be received as a Current Procedural Terminology (“CPT”) code. In some implementations, the information regarding the type of medical procedure performed may be in form of a text describing the procedure.

Upon receiving the information of the medical procedure of the patient 116b, the platform 104 searches the data storage 110 to retrieve a plurality of patient records that are associated with the medical procedure. For example, the plurality of patient records may include records of patients that were recommended for the same procedure and/or records of patients that have received the same medical procedure.

The platform 104 uses the Data processing (DP) engine 120 to extract relevant information from the patient records retrieved from the data storage 110. Information extracted from associated patient records may include, but is not limited to, patient diagnosis, patient demographics, the location the procedure was performed, length of operating room time used to perform the procedure, information regarding the medical personnel that performed the procedure, forms and other information obtained from the patient pre-procedure, equipment used to perform the procedure, post-procedure information obtained from the patient, and post-procedure medical complications.

The platform 104 uses the information extracted from the plurality of patient records to generate a questionnaire directed to one or more health issues associated with the medical procedure of the patient 116b. The DP engine 120 can apply machine learning techniques to generate questions of the questionnaire. For example, the DP engine 120 can determine the frequency with which item of information appear in the plurality of patient records and rank each item based on its prevalence. Based on this ranking, the DP engine 120 can generate a set of questions and/or notifications associated with the items of information and send the questions to the patient user 116b.

In some implementations, the plurality of patient records include one or more questions that the medical staff have previously asked when dealing with one or more procedures similar to the medical procedure of the patient 116b. The DP engine can determine questions that medical personnel asked most frequently and include those questions (or questions similar to the most frequently asked questions) in the questionnaire.

In some implementations, each of the questions included in the questionnaire are directed to one or more health categories. Some examples of health categories and corresponding questions that may be included in a questionnaire include, but are not limited to, wound condition (e.g., “Is your wound bleeding?”), medication consumption (e.g., “Have you had to take more medication than prescribed to manage pain?”), level of appetite (e.g., “Have you been eating?”), physical function (e.g., “Have you been able to go the restroom?”), vital signs (e.g., “What is your pulse?”, “What is your blood pressure?”), bodily discharges (“How often have you used the restroom in the past 6 hours?”), and pain level (e.g., “What is your level of pain on a scale of 1 to 10?”).

In some implementations, the questions on the questionnaire are generated based on one or more trends identified in one or more health categories associated with the plurality of patient records. The trends can be determined by applying machine learning technique on the plurality of patient records. In some examples, each question in the questionnaire is directed to one health category.

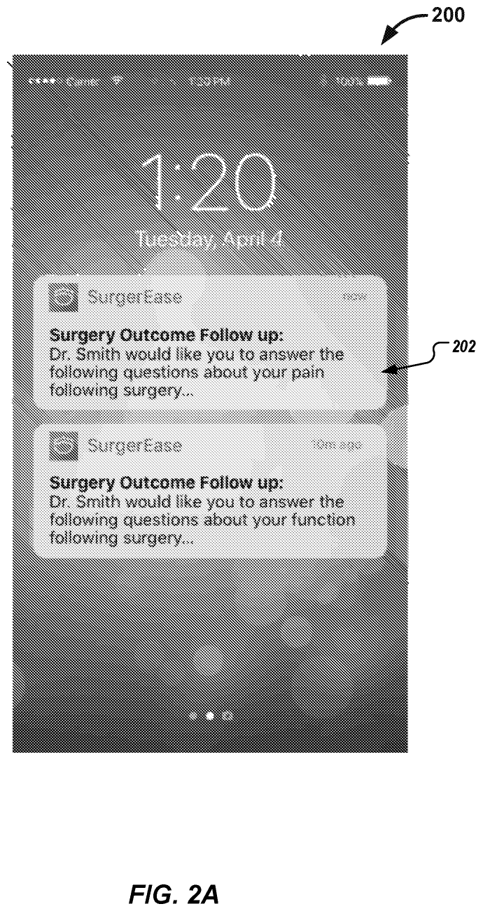

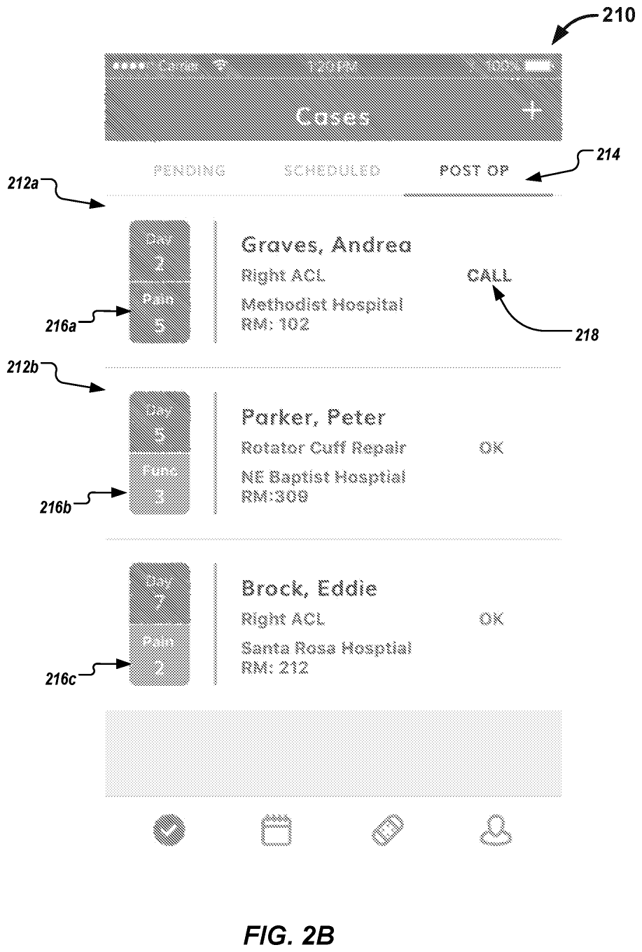

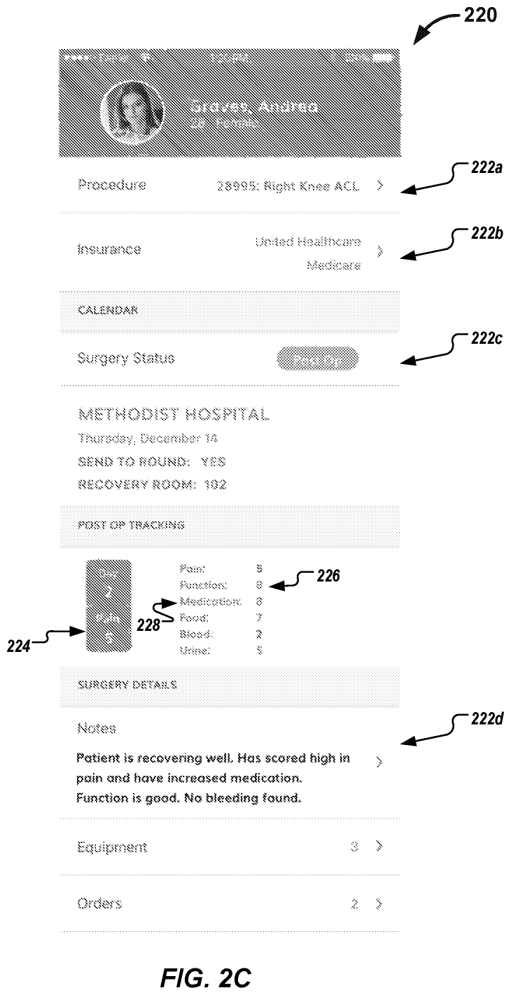

The platform 104 sends the questionnaire to a computing device associated with the patient 116b, for example, the user device 114b, through the user interface 112. The patient 116b answers the questions on the questionnaire and sends the responses back to the user interface 112. FIG. 2A depicts an example notification 202 on a computing device, e.g., the patient device 104b, requesting a patient to provide answers to a questionnaire.

The platform 104 uses the DP engine to review the answers received from the user device 114b and analyze the health condition of the patient 116b. Based on the health condition of the patient 116b, the DP engine determines whether to generate a notification to a medical staff and/or the patient 116b. For example, by comparing the answers to the plurality of medical records obtained from the data storage 110, the DP engine may predict that a health risk will likely to happen to the patient. The DP engine may provide a probability and/or a time of occurrence associated with the predicted health risk. As will be further described below, FIG. 2B provides a few example notifications displayed on a computing device, e.g., on the physician device 114c.

The DP engine communicates its decisions to the notifications engine 108. Depending on the content of the communications and predefined settings of the notification engine, the notification engine 108 may send one or more notifications to the patient's user device 114b, the physician's user device 114c, and/or the MA's user device 114a. The platform 104 may also use the rule engine 106 to schedule a proceeding (e.g., a surgery, an urgent injection, etc.) associated with the notification.

In some implementations, DP engine uses the answers to calculate a health score for the patient 116b. The health score can be used to determine what notification to be generated. For example, the platform 104 may provide a particular notification in response to determining that the health score is within a particular range. FIG. 2B, which is further described below, depicts different notifications 212a and 212b, respectfully associated with the health scores 216a and 216b. The notification engine 108 may provide different types or forms of notifications for the same health scores associated with two different procedures. For example, a notification for a health score of 7 for a post-surgery procedure may indicate a more urgent need for a medical attention than a health score of 7 for a post injection procedure.

In some implementations, if the health score generated by rules engine 106 for patient user 116b is within an acceptable range, notifications engine 108 sends a notification to a medical staff (e.g. to the physician 116c) indicating that patient user 116b is progressing well, and does not require medical attention at this time.

In some implementations, notifications engine 108 sends a notification to a medical staff only if the health score generated by rules engine 106 for patient user 116b is outside an acceptable range. For example, the health notification may be provided in response to determining that the health score is more than a predefined threshold value. The predefined threshold value may depend on the medical procedure of the patient 116b. For example, a first threshold value of 8 may be defined for a post injection medical procedure, and a second threshold value of 5 may be defined for a post surgery medical procedure.

The health score of the patient 116b can be calculated by comparing the answers submitted to the platform 104, with the information in the plurality of patient records obtained from the data storage 110. The information in the plurality of patient records that are compared to the answers can include, but is not limited to, answers to the same or similar treatment questions, diagnoses, number of post-treatment follow-up visits, treatment complications, post-treatment hospitalization records, and patient health scores. The health score generated for the patient 116b provides an indicator of the patient 116b current level of health.

In some implementations, DP engine 120 generates the health score of the patient 116b by using machine learning techniques to identify trends in one or more health categories associated with the plurality of patient records obtained from data storage 110. The DP engine 120 can generate the health score by comparing the answers to the questionnaire, to the trends identified in the one or more health categories.

In some implementations, the health score is calculated based on one of more sub-scores. A health score can be calculated by aggregating, averaging, or applying any other appropriate mathematical function on the one or more sub-scores. In some implementations, each sub-score is associated with one answer provided to a question of the questionnaire.

In some implementations, each sub-score can be associated with one or more health categories. For example, a sub-score associated with a particular health category can be determined by reviewing one or more answers that are provided to one or more questions (on the questionnaire) that are directed to the particular health category. The health score can be determined based on an aggregation of the one or more sub-scores associated with the one or more health categories. FIG. 2C, which is further described below, depicts an example notification that includes a health score 224 that is calculated based in the sub-scores 226.

The notification engine 108 can generate different forms of notifications in response to receiving different sub-scores from the DP engine 120. In some implementations, if a sub-score associated with one or more health categories is outside an acceptable range associated with the respective one or more health categories, notifications engine 108 sends a notification to the physician 116c to notify the physician about an urgent situation associated with the patient 116b. For example, if a sub-score associated with pain is outside an acceptable range for pain, notifications engine 108 send a notification to the user device 114c and/or user device 114a stating that the patient 116b requires medical attention.

An acceptable range may differ from one sub-score to another sub-score. For example, an acceptable range for a first sub-score can be between 0 and 5 while the acceptable range for a second sub-score is above 7. An acceptable range for a sub-score in one medical procedure can differ from an acceptable for the same sub-score in another medical procedure.

The notification engine 108 can send a notification to a medical staff whose specialty is associated with a health category corresponding to a sub-score that is outside a respective acceptable range. The notifications engine 108 can send a notification associated with the patient 116b to a medical staff who is assigned to the patient 116b, or whose specialty is associated with the medical procedure of the patient 116b. For example, the platform 104 can identify one or more on-call medical staff whose specialties are associated with the medical procedure of the patient 116b. The notification engine 108 can send the notification to at least one of the one or more on-call staff.

In some implementations, platform 104 can automatically schedule one or more medical procedures in response to the answers to the questionnaire. For example, the platform 104 may use the answers and/or the health score of the patient 116b to determine, based on the information obtained from the plurality of patient records obtained from the data storage 110, whether patient user 116b requires particular medical treatment procedure(s). The platform 104 can use the rules engine 106 to obtain information needed for scheduling the particular medical treatment procedure(s) and can automatically schedule the procedure(s).

For example, rules engine 106 can use machine learning to determine one or more medical personnel and/or particular medical specialties suitable for the particular medical treatment procedure(s). Notifications engine 108 can send one or more notifications to the patient 116b and/or one or more medical personnel (e.g., 116a, 116c) through their respective user devices, notifying them that the medical procedure has been scheduled.

FIGS. 2A-2C, 3A-3E, 4A-4F, 5A-5C, and 6A-6H depict examples of the notifications presented on a user device, according to the implementations of the present disclosure.

FIGS. 2A-2C depict examples of patient post-treatment tracking pages. FIG. 2A is an example patient user interface (UI) 200 for patient post-treatment tracking. The patient UI 200 provides the patient with post-treatment notifications 202, 204. These post-treatment patient notifications 202 can include a questionnaire with a series of questions for patient user to answer and submit to the platform. Responses provided to post-treatment notifications can be used to generate a health score that provides an indication of patient user's health, and potential need for further medical assistance. Post-treatment notifications can also include requests for documentation, requests for payment, or post-treatment appointment notifications.

FIG. 2B depicts an example physician user interface (UI) 210 for viewing the status of multiple patients. As shown in FIG. 2B, medical personnel, such as physicians, can monitor the status of their patients' cases through the notifications provided by the platform presented herein. In some implementations, each patient is associated with (e.g., assigned to) one or more medical staff, and the status of each patient appears as a notification 212 in the associated medical staff's UI 210.

In some implementations, medical personal can filter their cases by selecting a particular case status window 214. As shown in FIG. 2B, case status windows 214 can include pending, scheduled, and post-operative (“post-op”) cases. Medical personnel can monitor and receive information regarding their patients' condition through receipt of notifications 212.

Notifications 212 can include, but are not limited to, information regarding payment of medical bills, submissions of pre-treatment necessary documentation, patient diagnosis, location of treatment performance, patient's hospital room number, patient's contact information, physician notes, and the post-treatment condition of the patient.

In some implementations, one or more notifications 212a, 212b received by medical personnel include a respective health score 216a, 216b for the patients indicating the respective patients' health condition. In some implementations, notifications 212 received by medical personnel include one or more sub-scores for the patients indicating patients' condition as it relates to one or more associated health categories. Examples of sub-scores can include a function sub-score and a pain sub-score.

Notifications 212 can also identify follow-up actions 218 necessary for medical personnel to take. For example, follow-up actions 218 can notify the medical personnel the need to call the patient, schedule a follow-up treatment, or perform an emergency visit. In some implementations, follow-up actions are automatically generated based on the patient's health score and/or sub-scores. For example, a follow-up action 218 is included in the notification 212a because the health score 216a is considered outside an acceptable range associated with the respective patient's medical procedure.

FIG. 2C is an example physician user interface (UI) 220 for viewing an individual patient status. As shown, UI 220 includes individual patient information 222. Individual patient information can include, but is not limited to, patient name, age, gender, treatment procedure type, insurance information, surgery status, hospital information, patient's rounding status, and hospital room. In some implementations, UI 220 includes health scores 224 for the patient indicating specified patient's condition. The UI 220 includes one or more sub-scores 226 that indicate the specified patient's condition as it relates to one or more associated health categories 228.

In some implementations, medical personnel that are “on-call” at the specified patient's hospital can receive rounding notifications indicating that the patient has been sent to round and requires the on-call personnel to monitor the patient. In some implementations, the rounding notification is sent automatically to the on-call medical personnel based on the type of medical procedure specified for the individual patient. In some implementations, the rounding notification includes information regarding the physician who has requested the rounding notification. The rounding notification can include patient information, such as patient name, hospital room, and type of procedure performed. The rounding notification can include information for a plurality of patients requiring medical attention by the on-call medical personnel. In some implementations, patients will be automatically grouped based on procedure type and facility at which the procedure was performed, and an on-call medical personnel will automatically receive a rounding notification stating that all grouped patients require medical attention, and relevant information for all patients included in the group.

A user, such as an MA, can create a calendar event through the platform to schedule a procedure. The user can also specify personnel to be involved in the procedure, such as doctors, nurses, anesthesiologists, and so forth. Based on creation of the event, indicating when the surgery is to take place, the platform can automatically notify the relevant personnel who have been added to the event by the user. The personnel can respond yes or no to the notification, to indicate whether they are able to participate. The platform can collect the responses, and update the event accordingly. Those personnel who respond positively can be invited to view the event information through the various UIs of the platform. For example, on a case details screen for the procedure, the participants can view the pertinent information regarding the case (e.g., the surgical procedure). A physician or other user can log in to the platform and indicate that a particular piece of equipment is needed for the surgery. The MA or other user can fill in insurance information and/or other data regarding the patient. Through the platform, everyone involved in the surgery, including the doctor(s), the MA, the biller, the medical device representative, the anesthesiologist, the hospital, and the patient, can be “on the same page” and receive the relevant information and updates regarding the surgery, provide information regarding their roles or needs, receive notifications as appropriate, and so forth.

In some implementations, the platform includes various wizards to facilitate the start-up process for creating a new surgical event to be tracked through the platform. The wizard may help an MA, or other user, specify the team members to be invited to the case and be registered and associated with the surgery. The MA can create a new case, invite the relevant team members, and, once everyone has confirmed (including the patient), the MA can use the platform to move the case from a pending to a scheduled state. The involved personnel are then notified that the case is on, and the scheduled surgery event is added to their respective calendars. Through the calendar entry for a surgery, each individual can see everyone who is invited to participate, where the surgery is to be performed, what the particular procedure is, who is the patient, the status of the case (e.g., pending, scheduled, or post-op), and/or other relevant information. A check box coordination tool is provided to allow the MA, or other user, to follow their checklist process, to check off completed tasks, and thus track the various tasks that are to be performed before and after a surgery.

The platform also provides a patient view that communicates, to the patient, the information that is relevant to the surgery, including sending auto-reminder notifications for pre-surgery notes (e.g., stop eating 12 hours prior to surgery), and post-surgery notes to track pain levels, recovery status, and so forth. Such automated reminders (notifications) can be automatically sent to the patient, by the platform, through any appropriate communications channel(s), including emails, text messages, telephone voice messages, and so forth.

The platform improves and streamlines the workflows, processes, and communication that occurs among surgical care team members, such as surgeons, MAs, surgical coordinators (SCs), first assists, anesthesiologists, medical sales representatives, and so forth. The platform allows care team coordination through a shared calendar, and is designed to improve how the MA and/or SC communicates with the surgeon and the entire care team, as well as confirming the surgery time and location with their patients. The platform also allows for communication with the patient, including not only the ability to chat but the automated sending of notifications and reminders to the patient for all pre-op checklists, including billing and payment collection. After the procedure, the patient uses the platform to clearly understand their post-op instructions, order supplies and ask questions of the care team as well as posting their progress (including images when appropriate). The platform allows the surgeon and MA to closely and efficiently follow their patient's recovery in near real time, without the need to make phone calls.

In some implementations, the platform is designed to be most useful to the MA who coordinates information regarding the surgery. The platform helps the MA to save hours of time and become more efficient. Normally an MA spends hours of their time every week calling care team members and calling patients to coordinate schedules and/or answers questions. Because of these significant time savings provided through the platform, the MA is highly incentivized to sign up new patients on the platform, for example by helping them download the app and fully register and onboard the patient as a new user with a demo that is provided by the platform.

In addition to the foregoing benefits, the platform provides the following benefits to users such as a physician: save money in staff time by providing tools to enable the MA or SC to be more efficient in time management in the coordination of the surgery process; enhance the patient experience through complete and timely education, surgery information and instructions delivered in a timed approach; monitor the patient's progress through remote monitoring services and post-op pain and function push notifications; save money in call center fees with the patient messaging system; track patient communications with staff and care team; track billing progress, verify coding and ICD-10 within the program; verify that all persons and equipment are confirmed for the case; notify team of changes or needs for the case via messages or notes section of UIs; easy to track and manage surgery schedule with reminders; create post op patient rating system for each surgeon and procedure; and/or track physicians' surgeries by procedure and rank them against their fellow surgeons in the same specialty using specific metrics.

The platform can also provide the following benefits to users such as a MA and/or SC: reduce the amount of time it takes for staff to schedule and coordinate all the aspects of a surgery via an intuitive question and response wizard app; speed surgery creation with a smart app that will recreate the previous settings based on case selection and or insurance; creates a simple interface to provide the patient all of the pre-op and post-op instructions for surgery; verify that the patient has completed all of their pre-op requirements and have read all of their instructions with automated patient tracking and responses; notify the care team via email or push notifications and allows the team the ability to confirm through instant messaging; track upcoming surgeries by managing the case progression through Pending, Scheduled, and Post-Op states; communicate with the patient through in-app messaging without having to answer or return calls late in the day or during clinic; easily monitor on the progress of the post-op patient through Patient Progress tracking via notifications; and/or remind the patient of appointments, follow-ups, medications, rehab, etc. through automated in-app reminders and notifications.

Furthermore, the platform provides the following benefits to users such as a patient: the ability to easily communicate with the Care Team Staff; easily access all pre-op and post-op surgical instructions in one location; easily research what to expect during surgery via educational videos; read and acknowledge all surgery info and notices before surgery; push notifications for all reminders, schedule, medications, rehab, etc.; provide patients billing and co-pay info prior to surgery with ability to pay in app; access to post-op supplies and equipment; access prescriptions and request refills; Patient Progress tracking after surgery via real time notifications; and/or Patient Pain tracking after surgery via real time notifications.

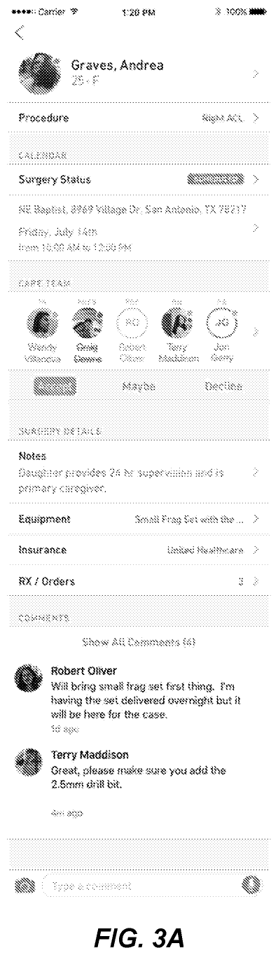

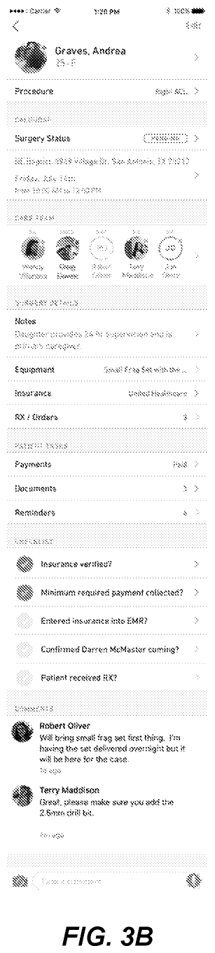

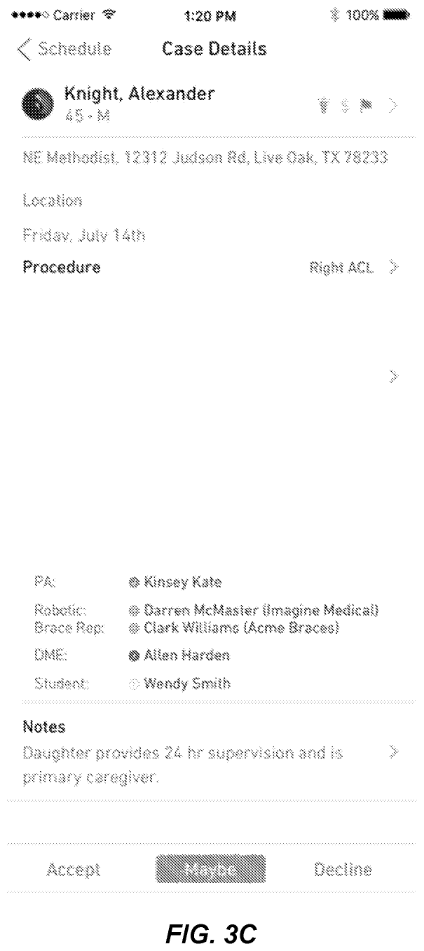

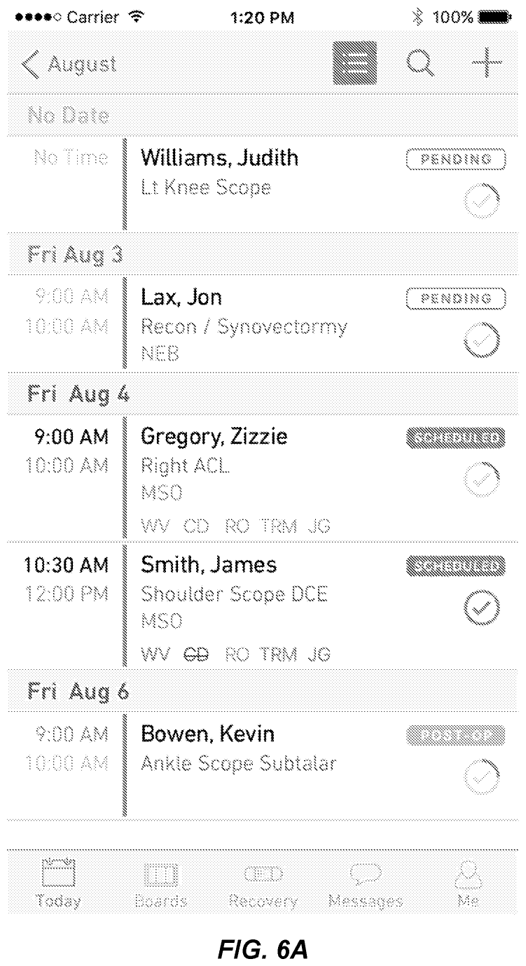

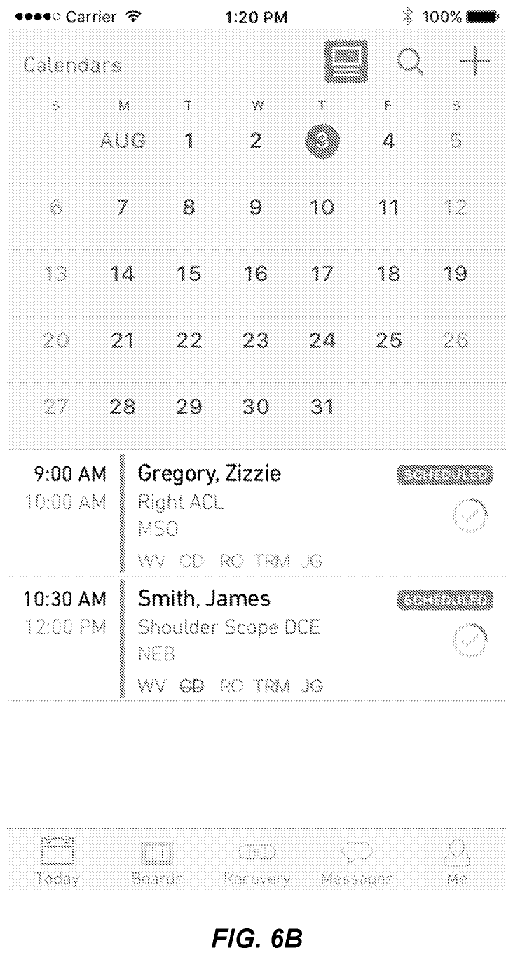

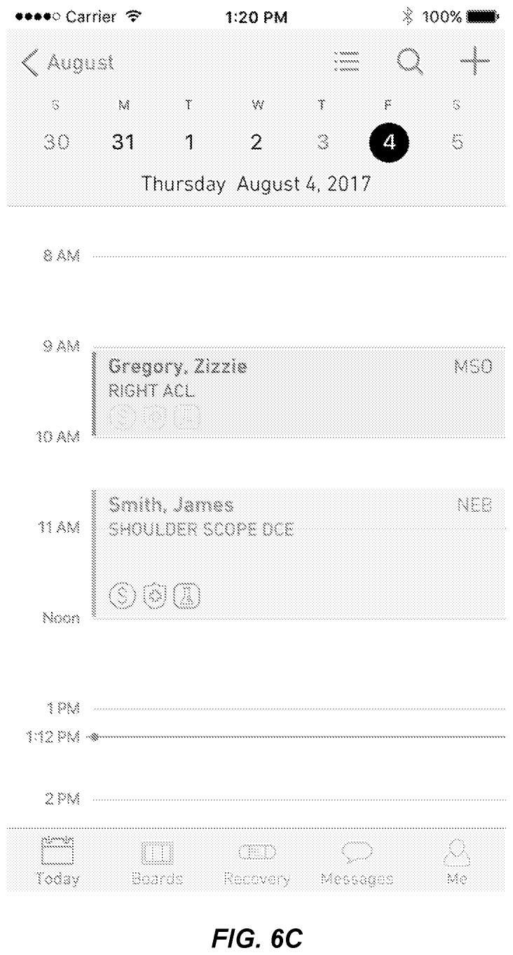

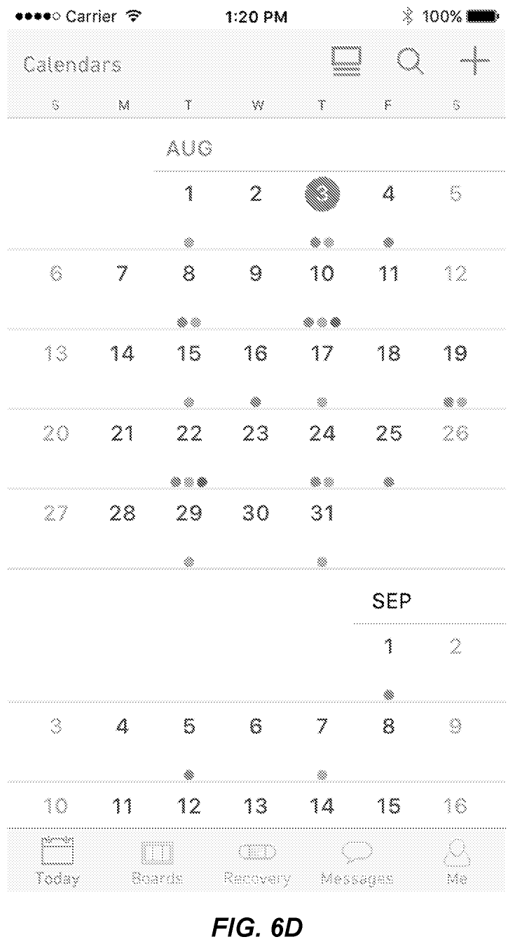

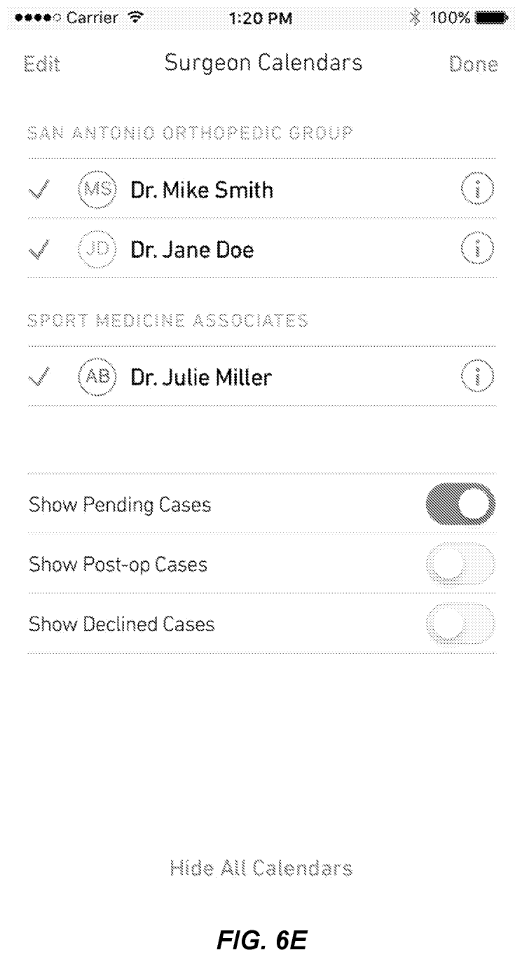

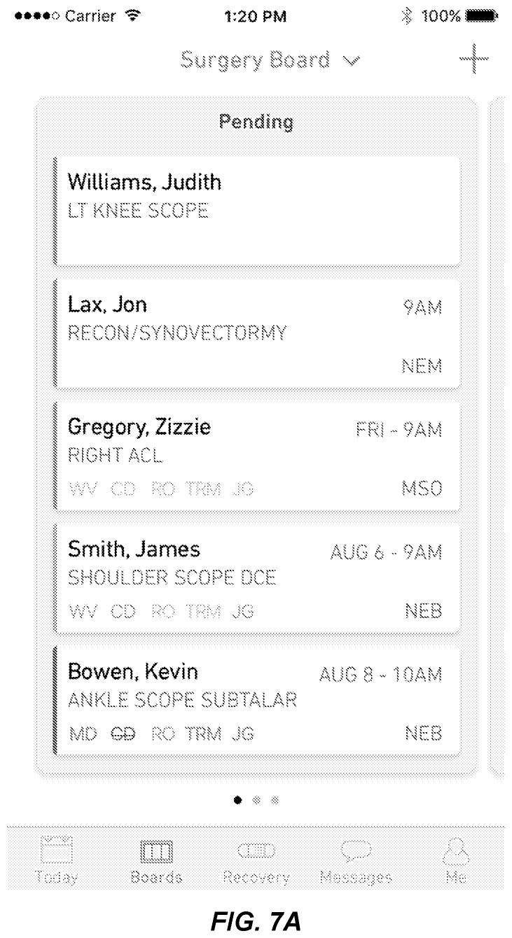

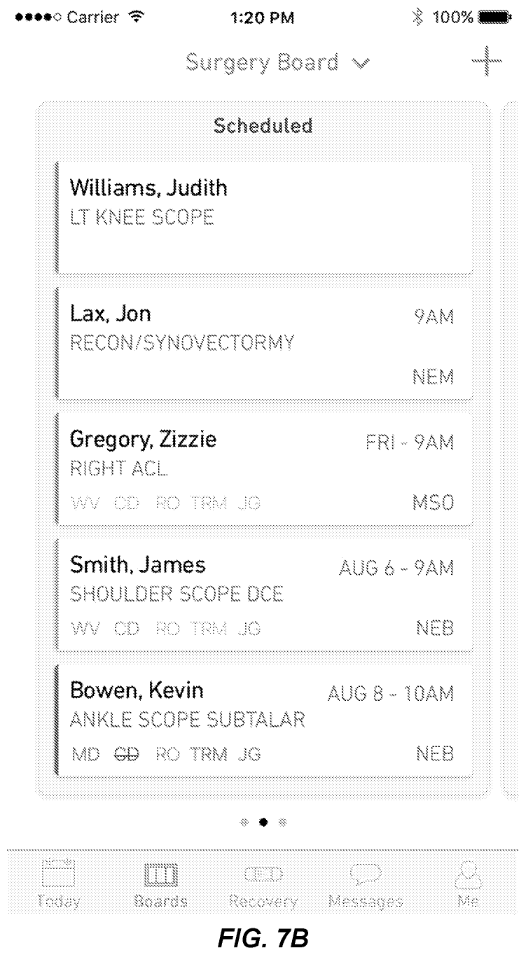

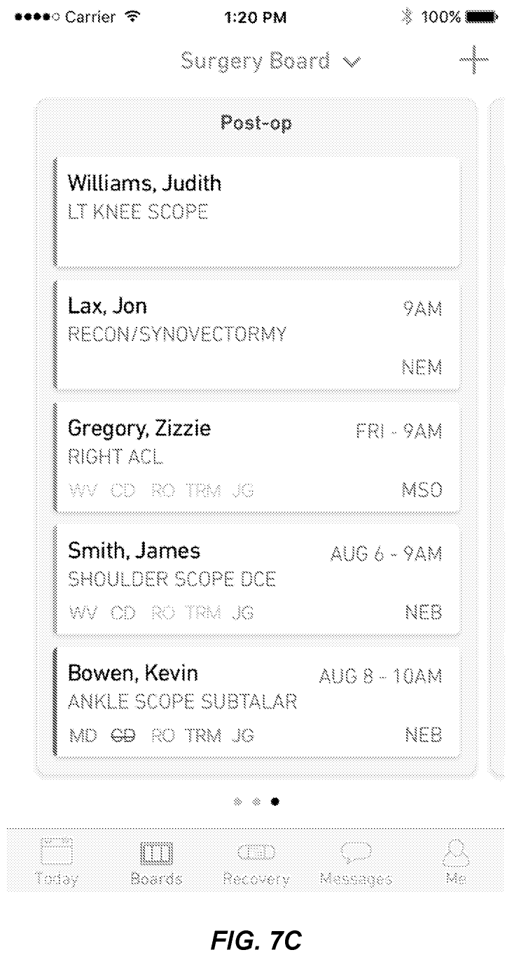

FIGS. 3A-3C are examples of case details pages that provide details regarding a case, such as a surgical procedure. FIGS. 6A-6D are examples of schedule calendar view pages for scheduling a case. FIGS. 7A-7C are examples of surgery board pages indicating the status of various cases. The UIs 112 may also include pages for welcoming a user to the platform, allowing a user to sign into the platform by providing credentials (e.g., username, password, etc.), and/or allowing a new user to register (e.g., sign up) with the platform by providing user information (e.g., name, username, role, credentials, contact information, etc.).

The UIs depicted in FIGS. 3A-3C allow a user to provide details for a case (e.g., a surgery), including verifying team members, indicating the type of surgery, the patient, and so forth. Patient information can be added to the case, including the patient's name, age, gender, date of birth, email address, telephone number, etc. The information for the procedure can be added under a surgery details section, through which a user can select a procedure (e.g., from a list), or enter a new procedure. On specification of the procedure, a wizard can auto-populate everything that was previously added by this user for a previous procedure of same type, such that the user can avoid adding those details to the newly specified procedure, such as team members, equipment, notes, etc.

The user can add notes about the procedure, such as the equipment needed, particular patient details, insurance information, prescriptions, orders, and so forth. Added information can trigger notifications to be sent. For example, added prescriptions can be sent directly to the patient, and/or orders can be sent directly to the team members (e.g., to have a wheelchair ready when patient gets out of surgery at a particular time).

Under the patient tasks section, information is presented regarding tasks to be performed by the patient, such as payments to be made, documents to be provided, and/or other reminders to patients. The patients may be automatically notified, and/or direct billing and payment may be processed through the platform. Documents may be presented to the patient to provide pre-op and/or post-op information, and the platform can track and confirm that the patient has read the documents. The particular documents provided may be automatically determined based on type of procedure, specialty, diagnosis and/or other information using machine learning from prior procedure and patient records.

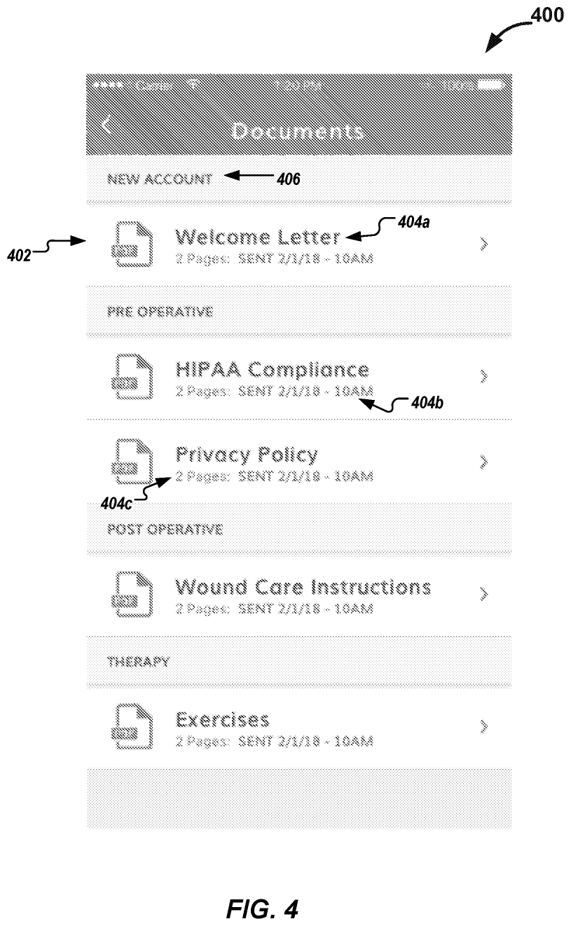

FIG. 4 depicts an example documents page 400 that allows users to view the documents 402 that the patient has provided. In some implementations, users can download the documents 402 directly for the documents page 400. In some implementations, the documents page provides detailed information 404 about each document, including, but not limited to, the document name, the number of page, the date the document was sent by the patient, and the time the document was sent patient. In some implementations, the documents are grouped by category 406. Document categories can include, but are not limited to, new account, preoperative, postoperative, and therapy.

The calendar status section of the view indicates the status of the case, whether it is in pending, scheduled, or post-op status. On the initial creation of a case, it is placed in pending status. During the pending status, the MA or other user can specify the information for the case, including team members, patient information, procedure details, etc. The user can place the case into scheduled status after all the requirements for the case have been satisfied. A case is moved to post-op status after the surgery is completed. Moving a surgery from pending to scheduled status causes various actions to be automatically performed by the platform, including notifying the team members, sending information to the patient, and so forth.

The views allow the user to specify location and date of the procedure, as well as the care team. The care team can communicate with one another, e.g., post questions, concerns, feedback, respond to same, and/or add comments, using a comments section of the views.

Through the reminder section, automated reminders can be scheduled to be sent as notifications to the patient. Such reminders may be scheduled to be sent at a time that is based on the surgery time, e.g., 12 hours before, 24 hours before, etc. Reminders can be sent after the surgery as well, to help track patient progress after the surgery, check how they are feeling, if they are experiencing pain, and so forth. For example, a post-op notification can be sent to ask the patient to rate their level of pain on a scale from 1-10. Follow-up questions can be asked depending on the answer to the initial pain rating. The patient's responses can be sent to the MA, surgeon, and/or others to let them track the patient's recovery progress without requiring phone calls.

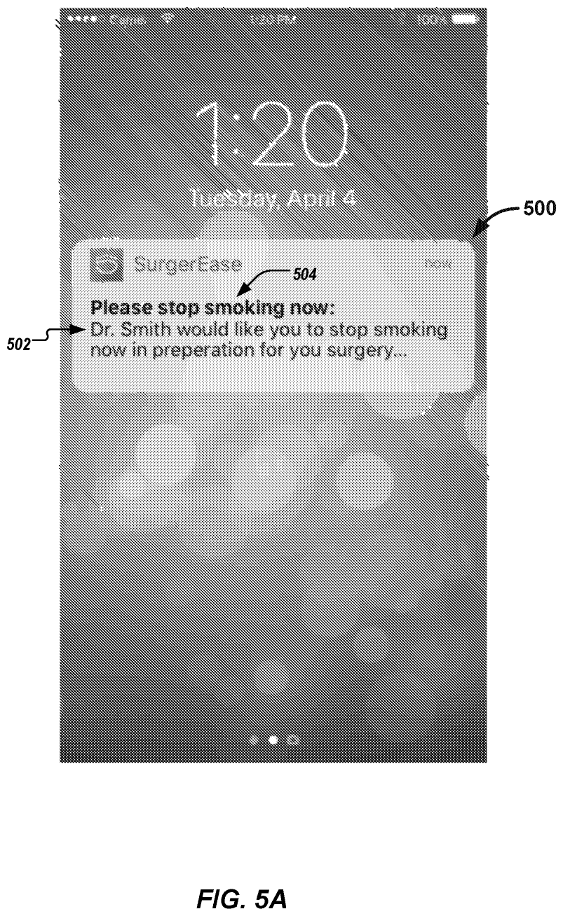

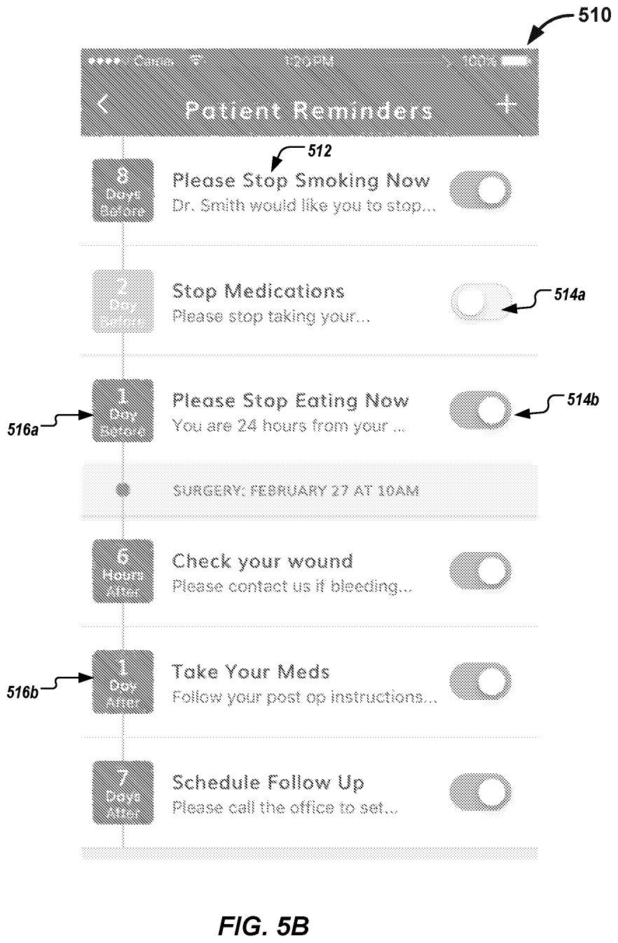

FIG. 5A is an example of a patient reminder notification 500. In some implementations, the patient reminder notification 500 includes the name of the medical personnel 502 associated with the patient user. The patient reminder notification interface 500 can include text specifying the action item(s) 504 that the patient should complete at the request of her associated medical personnel 502. FIG. 5B is an example patient reminder interface 510. In some implementations, patient can check off action items 512 as completed. In some implementations, patient can check off action items 512 as completed using toggle switch 514a, 514b in the notification interface 510. The patient reminder notification interface 510 can include the date and/or time 516a, 516b at which each action item 512 must be completed.

A checklist section can allow the MA or other user to keep track of a list of tasks to be performed prior to scheduling the surgery, such as verifying insurance, collecting any necessary payments, and so forth. The progress toward completing all the tasks can be tracked and presented in the calendar views as described below. Once all tasks in the checklist are completed, the case is cleared to be changed to status of scheduled, indicating that the surgery has been scheduled at the specific date/time and location, involving the personnel (team members) who have verified their availability and participation.

The calendar view pages, as shown in FIGS. 6A-6E, providing different calendar views for scheduled cases, such as daily, weekly, and monthly views as shown in the examples. In some instances, different patients (different cases) may be presented in different colors to help distinguish one case from another. In some views, as in the example of FIG. 6C, a case may be shown at a particular date and time, with icons to indicate prerequisites to be satisfied before scheduling the surgery. For example, in FIG. 6C, a case for James Smith is pending (e.g., a shoulder scope surgery), with three icons indicating that a copay, insurance verification, and lab work completion are to be performed as prerequisites prior to scheduling the case. The prerequisites may correspond to items in the checklist described above. All the boxes in the checklist are to be checked for the case to get approved for scheduling. In the calendar view, prerequisites may be presented differently based on whether they are completed or not yet completed.

In a calendar view, procedures may be presented with an indication of their current status (e.g., pending, scheduled, or post-op). The MA can use the platform to perform all the tasks to get the surgery scheduled while the surgery is in pending state. As shown in FIG. 6B, the case may be shown with an icon (e.g., the completion circle) that indicates the progress toward completion, from 0% to 100% complete. Once the circle is filled, indicating progress is complete, the check mark can change from gray to blue, indicating that the case is in a state that is ready to be scheduled, and all requisite tasks have been completed. In some implementations, tasks may be presented with different colors indicating different doctors.

Selecting a case from a calendar view page causes the case details page to be presented, as shown in FIGS. 3A-3C, to allow a user to drill down into more details regarding the case, update the details, check details status for prerequisites, notifications, patient tasks, and so forth.

In some implementations, surgeries can be automatically scheduled using machine learning based on previous surgeries and pre-programmed defaults. For example, after a MA specifies a type of procedure to be performed, a wizard can auto-populate and schedule required items for the procedure. Items automatically scheduled can include, but are not limited to, length of operating room time, any necessary medical equipment, number of technicians required, medications, and recovery rooms. In some implementations, the machine learning is performed on patient records associated with the specified procedure and pre-programmed defaults stored on a database to automatically determine scheduling requirements. In some implementations, specific medical personnel assigned to a scheduled surgery will automatically receive notifications if surgery requirements directly relating to the specific personnel have changed. For example, a surgical device representative assigned to a scheduled surgery will automatically receive a notification if the surgery is changed to no longer involve the device the representative is representing.

The surgery boards pages of FIGS. 7A, 7B, and 7C list cases that are in pending, scheduled, and post-op status, respectively. The surgery boards pages provide a quick view of where each patient is at in pending, scheduled, or post-op status. In some implementations, a user can also create new boards based on workflow, e.g., to provide a custom workflow and tracking mechanism. In this way, implementations provide an open environment with configurable workflows that can be tracked and/or monitored by team members or other users. A particular case (e.g., surgery) moves from one screen to another as its status changes from pending to scheduled, and then from scheduled to post-op.

In some implementations, the UIs also include recovery view page(s) through which a patient can enter information regarding their recovery, rate of pain, and so forth. The page(s) can also allow a patient to upload images of their wound, incision, and so forth, and/or other information that is sent to the MA and/or physician, to help them track the patient's recovery and follow up as appropriate. The UIs can also include a messages page to let the various users compose, send, and view messages sent between users.

In some implementations, the platform provides UI(s) that allow patients to rank and/or rate their physicians and/or other team members. Patients can rate or otherwise review the level of service they have received. Such information can be aggregated among patients and/or cases, and the platform can rank physicians according to their types of procedures performed and/or their specialties, in a geographic region and/or generally.

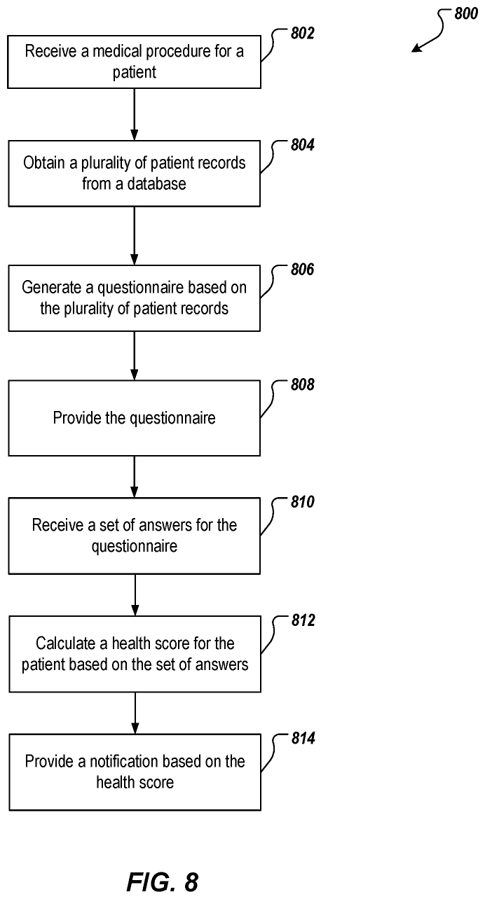

FIG. 8 depicts an example process 800 that can be executed in accordance with implementations of the present disclosure. In some implementations, the example process 800 may be performed using one or more computer-executable programs executed using one or more computing devices. The process can be performed using an application platform, such as the exemplary platform 104 illustrated in FIG. 1.

Medical procedure information associated with a patient is received (802). Medical procedure information received by the platform can include, but is not limited to, information related to pre-operative treatment procedures, surgical procedures, and post-operative treatment procedures. In some implementations, the information regarding the type of medical procedure performed is received as a Current Procedural Terminology (“CPT”) code. In other implementations, the information regarding the type of medical procedure performed is received as text describing the procedure.

A plurality of patient records are obtained from a database (804). The plurality of patient records retrieved can be limited to those records associated with the specified medical procedure received at 802. For example, associated patient records may include records of patients that were recommended for the same procedure and records of patients that received the same procedure.

A questionnaire is generated based on the plurality of patient records (806). The questionnaire can be generated based on machine learning techniques, as described earlier in this disclosure. The questions included in the questionnaire can each be directed to one or more health categories.

The questionnaire is provided (808). For example, the questionnaire can be sent to a user device associated with the patient. The patient accesses the questionnaire through a user interface on the user device.

A set of answers are received are received for the questionnaire (810). For example, the patient can answer the questions of the questionnaire on the user device. The answers can be in the form of text strings, yes/no answers, or numbers.

A health score is calculated for the patient based on the set of answers received (812). For example, the platform 104 can generate the patient health score by comparing the questionnaire answers submitted by the patient user to information in the plurality of patient records obtained from the database. By using machine learning techniques to compare the questionnaire responses obtained from the patient user with the information obtained from the associated patient records stored on the database, the platform can generate the health score associated with the patient. The health score can be an indicator of the patient user's current level of health. In some implementations, the platform generates patient user's health score by, at least in part, using machine learning to identify trends in one or more health categories associated with the plurality of patient records obtained from the database. In some implementations, the platform generates patient user's health score by, at least in part, comparing patient user's questionnaire responses to trends identified in one or more health categories associated with the plurality of patient records obtained from the database. In some implementations, the health score provides predictive information about the patient user's level of risk of experiencing a medical emergency or post-operative complication at a future time.

A notification to based on the health score is provided (814). For example, if the health score(s) for patient user is outside an acceptable range, a notification may be sent to a physician's user device stating that patient user requires medical attention. If the health score(s) is within an acceptable range, a notification may be sent to the physician's user device stating that patient user is progressing well, and does not require medical attention at this time.

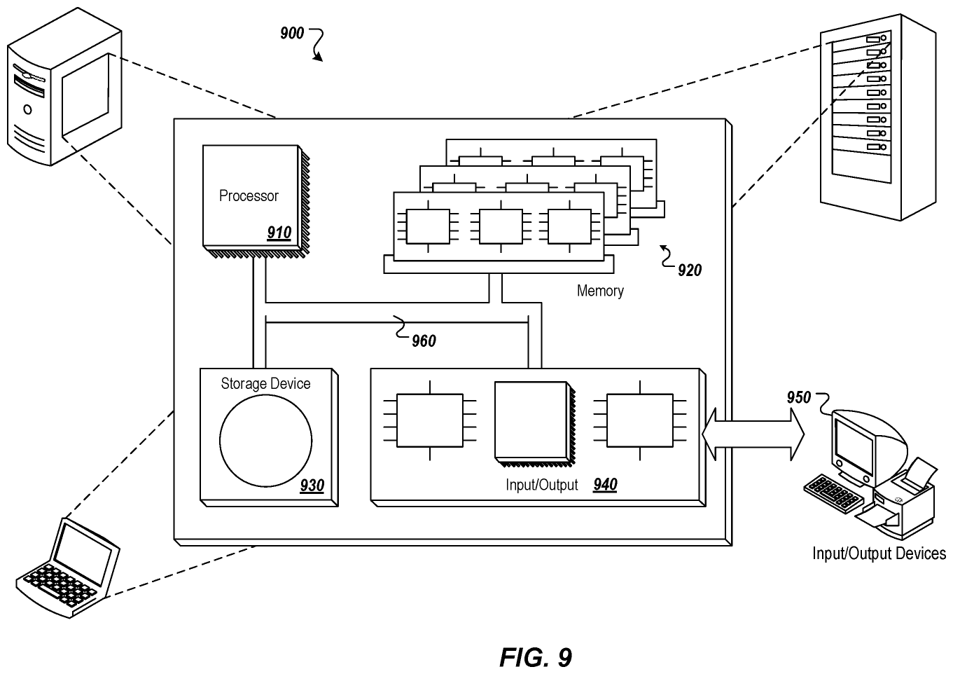

FIG. 9 depicts an example computing system, according to implementations of the present disclosure. The system 900 may be used for one or more of the operations described with respect to the various implementations discussed herein. For example, the system 900 may be included, at least in part, in one or more of the user device 102, the management device(s) 108, or other computing device(s) described herein. The system 900 may include one or more processors 910, a memory 920, one or more storage devices 930, and one or more input/output (I/O) devices 950 controllable through one or more I/O interfaces 940. The various components 910, 920, 930, 940, or 950 may be interconnected through at least one system bus 960, which may enable the transfer of data between the various modules and components of the system 900.

The processor(s) 910 may be configured to process instructions for execution within the system 900. The processor(s) 910 may include single-threaded processor(s), multi-threaded processor(s), or both. The processor(s) 910 may be configured to process instructions stored in the memory 920 or on the storage device(s) 930. The processor(s) 910 may include hardware-based processor(s) each including one or more cores. The processor(s) 910 may include general purpose processor(s), special purpose processor(s), or both.

The memory 920 may store information within the system 900. In some implementations, the memory 920 includes one or more computer-readable media. The memory 920 may include any suitable number of volatile memory units and/or non-volatile memory units. The memory 920 may include read-only memory, random access memory, or both. In some examples, the memory 920 may be employed as active or physical memory by one or more executing software modules.

The storage device(s) 930 may be configured to provide (e.g., persistent) mass storage for the system 900. In some implementations, the storage device(s) 930 may include one or more computer-readable media. For example, the storage device(s) 930 may include a floppy disk device, a hard disk device, an optical disk device, or a tape device. The storage device(s) 930 may include read-only memory, random access memory, or both. The storage device(s) 930 may include one or more of an internal hard drive, an external hard drive, or a removable drive.

One or both of the memory 920 or the storage device(s) 930 may include one or more computer-readable storage media (CRSM). The CRSM may include one or more of an electronic storage medium, a magnetic storage medium, an optical storage medium, a magneto-optical storage medium, a quantum storage medium, a mechanical computer storage medium, and so forth. The CRSM may provide storage of computer-readable instructions describing data structures, processes, applications, programs, other modules, or other data for the operation of the system 900. In some implementations, the CRSM may include a data store that provides storage of computer-readable instructions or other information in a non-transitory format. The CRSM may be incorporated into the system 900 or may be external with respect to the system 900. The CRSM may include read-only memory, random access memory, or both. One or more CRSM suitable for tangibly embodying computer program instructions and data may include any suitable type of non-volatile memory, including but not limited to: semiconductor memory devices, such as EPROM, EEPROM, and flash memory devices; magnetic disks such as internal hard disks and removable disks; magneto-optical disks; and CD-ROM and DVD-ROM disks. In some examples, the processor(s) 910 and the memory 920 may be supplemented by, or incorporated into, one or more application-specific integrated circuits (ASICs).

The system 900 may include one or more I/O devices 950. The I/O device(s) 950 may include one or more input devices such as a keyboard, a mouse, a pen, a game controller, a touch input device, an audio input device (e.g., a microphone), a gestural input device, a haptic input device, an image or video capture device (e.g., a camera), or other devices. In some examples, the I/O device(s) 950 may also include one or more output devices such as a display, LED(s), an audio output device (e.g., a speaker), a printer, a haptic output device, and so forth. The I/O device(s) 950 may be physically incorporated in one or more computing devices of the system 900, or may be external with respect to one or more computing devices of the system 900.

The system 900 may include one or more I/O interfaces 940 to enable components or modules of the system 900 to control, interface with, or otherwise communicate with the I/O device(s) 950. The I/O interface(s) 940 may enable information to be transferred in or out of the system 900, or between components of the system 900, through serial communication, parallel communication, or other types of communication. For example, the I/O interface(s) 940 may comply with a version of the RS-232 standard for serial ports, or with a version of the IEEE 1284 standard for parallel ports. As another example, the I/O interface(s) 940 may be configured to provide a connection over Universal Serial Bus (USB) or Ethernet. In some examples, the I/O interface(s) 940 may be configured to provide a serial connection that is compliant with a version of the IEEE 1394 standard.

The I/O interface(s) 940 may also include one or more network interfaces that enable communications between computing devices in the system 900, or between the system 900 and other network-connected computing systems. The network interface(s) may include one or more network interface controllers (NICs) or other types of transceiver devices configured to send and receive communications over one or more networks using any suitable network protocol.

Computing devices of the system 900 may communicate with one another, or with other computing devices, using one or more networks. Such networks may include public networks such as the internet, private networks such as an institutional or personal intranet, or any combination of private and public networks. The networks may include any suitable type of wired or wireless network, including but not limited to local area networks (LANs), wide area networks (WANs), wireless WANs (WWANs), wireless LANs (WLANs), mobile communications networks (e.g., 3G, 4G, Edge, etc.), and so forth. In some implementations, the communications between computing devices may be encrypted or otherwise secured. For example, communications may employ one or more public or private cryptographic keys, ciphers, digital certificates, or other credentials supported by a security protocol, such as any version of the Secure Sockets Layer (SSL) or the Transport Layer Security (TLS) protocol.

The system 900 may include one or more computing devices of any suitable type. The computing device(s) may include, but are not limited to: a personal computer, a smartphone, a tablet computer, a wearable computer, an implanted computer, a mobile gaming device, an electronic book reader, an automotive computer, a desktop computer, a laptop computer, a notebook computer, a game console, a home entertainment device, a network computer, a server computer, a mainframe computer, a distributed computing device (e.g., a cloud computing device), a microcomputer, a system on a chip (SoC), a system in a package (SiP), and so forth. Although examples herein may describe computing device(s) as physical device(s), implementations are not so limited. In some examples, a computing device may include one or more of a virtual computing environment, a hypervisor, an emulation, or a virtual machine executing on one or more physical computing devices. In some examples, two or more computing devices may include a cluster, cloud, farm, or other grouping of multiple devices that coordinate operations to provide load balancing, failover support, parallel processing capabilities, shared storage resources, shared networking capabilities, or other aspects.

Implementations and all of the functional operations described in this specification may be realized in digital electronic circuitry, or in computer software, firmware, or hardware, including the structures disclosed in this specification and their structural equivalents, or in combinations of one or more of them. Implementations may be realized as one or more computer program products, i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter effecting a machine-readable propagated signal, or a combination of one or more of them. The term “computing system” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any appropriate form of programming language, including compiled or interpreted languages, and it may be deployed in any appropriate form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and/or processor(s) of any appropriate kind of digital computer. Generally, a processor may receive instructions and data from a read only memory or a random access memory or both. Elements of a computer can include a processor for performing instructions and one or more memory devices for storing instructions and data. Generally, a computer may also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, implementations may be realized on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any appropriate form of sensory feedback, e.g., visual feedback, auditory feedback, or tactile feedback; and input from the user may be received in any appropriate form, including acoustic, speech, or tactile input.

Implementations may be realized in a computing system that includes a back end component, e.g., as a data server, or that includes a middleware component, e.g., an application server, or that includes a front end component, e.g., a client computer having a graphical UI or a web browser through which a user may interact with an implementation, or any appropriate combination of one or more such back end, middleware, or front end components. The components of the system may be interconnected by any appropriate form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some examples be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

What is claimed is:

1. A computer-implemented method executed by a system comprising one or more processors, the method comprising:
receiving, by the system, patient information comprising a medical procedure associated with a patient;
obtaining, by the system and from a database, a plurality of patient records associated with the medical procedure, the plurality of patient records including medical histories of a plurality of patients;
generating, by the system, a questionnaire by applying a machine learning technique on the plurality of patient records, the questionnaire comprising a set of questions derived from the plurality of patient records;
providing, by the system, the questionnaire to the patient;
receiving, by the system, a set of answers for the set of questions;
calculating a health score for the patient, the health score being calculated based on the set of answers and the plurality of patient records; and
providing, by the system and based on the health score, a health notification associated with the patient, the health notification is being provided to be displayed on a computing device.

2. The method of claim 1, wherein the health notification includes a prediction of a health risk that is to happen to the patient at a future time.

3. The method of claim 1, wherein the set of questions are generated based on one or more trends identified in one or more health categories associated with the plurality of patient records, the one or more trends being determined by applying the machine learning technique on the plurality of patient records.

4. The method of claim 3, wherein the health score is calculated by comparing the set of answers to the one or more trends.

5. The method of claim 1, wherein the health notification is provided in response to determining that the health score is more than a predefined threshold value.

6. The method of claim 1, wherein the set of questions are directed to one or more health categories and the health score is calculated based on an aggregation of one or more sub-scores associated with the one or more health categories.

7. The method of claim 6, wherein the health notification is provided in response to determining that a sub-score of the one or more sub-scores is beyond a predefined range associated with a respective health category that corresponds with the sub-score.

8. The method of claim 7, wherein the health notification is provided to a medical staff whose specialty is associated with the respective health category.

9. The method of claim 6, wherein at least one health category of the one or more health categories is associated with at least one of a pain level, physical functions, a history of medications consumption, level of appetite, and vital signs of the patient.

10. The method of claim 6, wherein each question in the set of questions is directed to one health category of the one or more health categories.

11. The method of claim 6, wherein each sub-score is associated with one answer in the set of answers.

12. The method of claim 1, wherein the patient is associated with one or more medical staff, and the computing device on which the health notification is being displayed is associated with at least one of the one or more medical staff

13. The method of claim 1, wherein the health notification is provided to an on-call medical staff whose specialty is related to the medical procedure.

14. The method of claim 1, wherein the medical procedure includes one or more procedures based on which the patient is currently being treated.

15. The method of claim 1, wherein the medical procedure includes post-surgical treatment procedures.

16. The method of claim 1, wherein the medical procedure includes pre-surgical treatment procedures.

17. The method of claim 1, further comprising automatically scheduling one or more treatment procedures based on the health score and the medical histories in the plurality of patient records.

18. A non-transitory, computer-readable medium storing one or more instructions executable by a computer system to perform operations comprising:
receive patient information comprising a medical procedure associated with a patient;
obtain, from a database, a plurality of patient records associated with the medical procedure, the plurality of patient records including medical histories of a plurality of patients;
generate a questionnaire by applying a machine learning technique on the plurality of patient records, the questionnaire comprising a set of questions derived from the plurality of patient records;
provide the questionnaire to the patient;
receive a set of answers for the set of questions;
calculate a health score for the patient, the health score being calculated based on the set of answers and the plurality of patient records; and
provide, based on the health score, a health notification associated with the patient, the health notification is being provided to be displayed on a computing device.

19. The non-transitory computer-readable medium of claim 18, wherein the health notification includes a prediction of a health risk that is to happen to the patient at a future time.

20. A computer-implemented system, comprising:
a computer memory; and
a hardware processor interoperably coupled with the computer memory and configured to perform operations comprising:
receive patient information comprising a medical procedure associated with a patient;
obtain, from a database, a plurality of patient records associated with the medical procedure, the plurality of patient records including medical histories of a plurality of patients;
generate a questionnaire by applying a machine learning technique on the plurality of patient records, the questionnaire comprising a set of questions derived from the plurality of patient records;
provide the questionnaire to the patient;
receive a set of answers for the set of questions;
calculate a health score for the patient, the health score being calculated based on the set of answers and the plurality of patient records; and
provide, based on the health score, a health notification associated with the patient, the health notification is being provided to be displayed on a computing device.
